(19) United States
(12) Patent Application Publication
(10) Pub. No.: US 2013/0060004 A1
(43) Pub. Date: Mar. 7, 2013

(54) Novel Process For The Preparation Of Leuprolide And Its Pharmaceutically Acceptable Salts Thereof

(71) Applicants: Ananda Kuppanna, Hyderabad (IN); Bulli Raju Kamana, Hyderabad (IN); Sreelatha Vanjivaka, Hyderabad (IN); Debashish Datta, Hyderabad (IN)

(72) Inventors: Ananda Kuppanna, Hyderabad (IN); Bulli Raju Kamana, Hyderabad (IN); Sreelatha Vanjivaka, Hyderabad (IN); Debashish Datta, Hyderabad (IN)

(21) Appl. No.: 13/696,656

(22) PCT Filed: May 4, 2011
(86) PCT No.: PCT/IN11/00312

§ 371 Date: Nov. 7, 2012

(30) Foreign Application Priority Data
May 7, 2010 (IN) 1278/CHE/20110

(51) Int. Cl. C07K 1/10; C07K 1/14; C07K 7/08

(52) U.S. Cl. 530/328

(57) ABSTRACT

The present invention relates to a novel process for the preparation of Leuprolide or its pharmaceutically acceptable salts thereof by solid and solution phase peptide synthesis (Hybrid approach). The present invention also relates to a process for the preparation of Leuprolide or its pharmaceutically acceptable salts thereof by synthesizing the peptide fragments by solid phase (7 and 5 amino acids fragment) and solution phase (2 and 4 amino acids fragment) respectively. The final solution phase condensation of these peptide fragments (7+2 and 5+4) led to a nonapeptide Leuprolide in the protected form. The present invention further relates to novel peptide fragements—Pyr-His(Trt)-Trp(Boc)-Ser(tBu)-Tyr(tBu)-DLeu-Leu-OH (Fragment-11); H-Arg(Pbf)-Pro-NHEt (Fragment-I11); Pyr-His(Trt)-Trp(Boc)-Ser(tBu)-Tyr(tBu)-DLeu-Leu-Arg(Pbf)-Pro-NHEt (Protected Leuprolide) (Fragment-IV); Pyr-His(Trt)-Trp(Boc)-Ser(tBu)-Tyr(tBu)-OH (Fragment-V); H-DLeu-Leu-Arg(Pbf)-Pro-NHEt (Fragment-VI) and process for the preparation thereof.

This application claims priority to Indian patent application 1278/CHE/2010 filed on May 7, 2010

### FIELD OF THE INVENTION

The present invention relates to a novel process for the preparation of Leuprolide or its pharmaceutically acceptable salts thereof by solid and solution phase peptide synthesis (Hybrid approach).

The present invention also relates to a process for the preparation of Leuprolide or its pharmaceutically acceptable salts thereof by synthesizing the peptide fragments by solid phase (7 and 5 amino acids fragment) and solution phase (2 and 4 amino acids fragment) respectively. The final solution phase condensation of these peptide fragments (7+2 and 5+4) led to a nonapeptide Leuprolide in the protected form.

The present invention further relates to novel peptide fragements—Pyr-His(Trt)-Trp(Boc)-Ser(tBu)-Tyr(tBu)-DLeu-Leu-OH (Fragment-II); H-Arg(Pbf)-Pro-NHEt (Fragment-III); Pyr-His(Trt)-Trp(Boc)-Ser(tBu)-Tyr(tBu)-DLeu-Leu-Arg(Pbf)-Pro-NHEt (Protected Leuprolide) (Fragment-IV); Pyr-His(Trt)-Trp(Boc)-Ser(tBu)-Tyr(tBu)-OH (Fragment-V); H-DLeu-Leu-Arg(Pbf)-Pro-NHEt (Fragment-VI) and process for the preparation thereof.

### BACK GROUND OF THE INVENTION

Leuprolide acetate belongs to the general class of drugs known as hormone agonists. It is a synthetic nonapeptide similar to the luteinizing hormone releasing hormone (LHRH) having a DLeu at position 6 in the place of glycine of the native hormone, that regulates the production of follicle-stimulating hormone (FSH) and luteinizing hormone (LH) produced by the pituitary gland. Leuprolide acetate is a potent gonadotropin releasing hormone receptor (GnRHR) agonist used for diverse clinical applications, including the treatment of prostate cancer, endometriosis, uterine fibroids, and central precocious puberty.

Leuprolide can be represented in terms of chemical formula as follows:

### 5-oxo-L-prolyl-L-histidyl-L-tryptophyl-L-seryl-L-tyrosyl-D-leucyl-L-leucyl-L-arginyl-N-ethyl-L-prolinamide

The nonapeptide leuprolide or leuprorelin is a synthetic peptide having strong ovulation inducing activity, and is known since many years. The compound as well as derivatives thereof are described for instance in DE 2446005C2, 3914412 or U.S. Pat. No. 4,005,063 using Merrifield resin in a 25% overall yield.

At present, two methods have been reported for the preparation of C-terminal peptide N-ethylamide: i) the classical solution method which involves a multistep cleavage and deprotection procedure; and ii) the classical solid-phase method, using Boc/Benzyl chemistry either directly on alkylated resin amino ethyl resin (J. Rivier, et al, IJPPR (1985) 25:414-420) or indirectly on Merrifield resin followed by aminolysis where the C-terminus amino acid of the peptide is attached to the solid support by an ester bond (D. H. Coy et al., Biochemistry (1974) 13:303). Both the direct method and indirect method however are some draw backs. With the direct method, peptide yield is relatively low after cleavage. The indirect method is not applicable to peptides containing Asp and/or Glu residues in which the additional carboxyl group is protected as the benzyl ester, which will also undergo aminolysis by the amine, resulting in the formation of Asn(N-alkylamide) and Glu(N-alkylamide) slow reaction times results in racemization. More over, Boc-SPPS generally utilizes hazardous reagents such as HF.

To overcome the problems associated with BocSPPS synthesis of peptides, Fmoc-SPPS has gained in popularity because of its use of environmentally safer reagents and comparatively milder reaction conditions. In particular, Fmoc-SPPS peptide synthesis avoids the use of HF. One resin that can be utilized for the Fmoc-SPPS synthesis of peptides with secondary amides is a resin bound amine (A. A. Zompra, et al., Chemistry Today (2006) 24(4):49-51). This resin has also been successfully used for the synthesis of small organic molecules such as secondary amides, ureas, sulfonamides, and guanidines (K. G. Estep, et al., J. Org. Chem. (1998) 63:5300-5301). Amine bound resins, however, have several drawbacks for Fmoc-SPPS synthesis of peptides. For example, the synthesis of secondary amides has been reported to be difficult. These supports also require expensive intermediates, multi-step reactions, and/or longer reaction time. Taken together, currently available amine bound supports are generally not practical or economical for the large-scale production of peptides with secondary amides.

Therefore, there exists a need to develop an alternate and improved process for the preparation of Leuprolide with high yield and purity. Further the process involved should be simple, convenient and cost-effective for large scale production.

### SUMMARY OF THE INVENTION

The main aspect of the present invention is to provide a process for the preparation of Leuprolide or its pharmaceutically acceptable salts thereof by synthesizing the peptide fragments by solid phase (7 and 5 amino acids fragment) and solution phase (2 and 4 amino acids fragment) respectively. The final solution phase condensation of these peptide fragments (7+2 and 5+4) led to a nonapeptide Leuprolide in the protected form.

Another aspect of the present invention is to provide novel peptide fragments Pyr-His(Trt)-Trp(Boc)-Ser(tBu)-Tyr(tBu)-DLeu-Leu-OH (Fragment-II); H-Arg(Pbf)-Pro-NHEt (Fragment-III); Pyr-His(Trt)-Trp(Boc)-Ser(tBu)-Tyr(tBu)-DLeu-Leu-Arg(Pbf)-Pro-NHEt (Protected Leuprolide) (Fragment-IV); Pyr-His(Trt)-Trp(Boc)-Ser(tBu)-Tyr(tBu)-OH (Fragment-V); H-DLeu-Leu-Arg(Pbf)-Pro-NHEt (Fragment-VI) and process for preparation thereof.

Yet another aspect of the present invention is to provide a process for the preparation seven amino acids fragment (Fragment-II) or five amino acids fragment (Fragment-V) by solid phase synthesis to yield the peptide of interest. Generally, the methods of the invention comprise C-terminal amino acid of the seven amino acids fragment (Fragment—II) or five amino acids fragment (Fragment-V) fragments is loaded on to the 2-CTC resin in DMF in presence of excess of DIEA. Unreacted functional sites are capped with methanol in presence of DIEA The fragment is then built using standard solid phase chemistry by first removing the N-terminal Fmoc group with piperidine in DMF and then adding a solution of the next Fmoc amino acid that has been pre-activated with DIC/6-CI-HOBt in DMF. The fragment is then cleaved from the resin using cold 1% TFA in DCM. The TFA is quenched with DIEA, and the DCM is evaporated. The peptide fragments are precipitated by the addition of DCM/IPE and isolated in high yield and purity.

The solution phase assembly of Fragment-III begins with the coupling of H-Pro-NHEt to the C-terminus of Fmoc-Arg(Pbf)-OH in DMF using HBTU/6-CI-HOBt/DIEA. The Fmoc-group is removed in situ and the amine product is isolated by precipitation with MTBE. After washing thoroughly the H-Arg(Pbf)-Pro-NHEt is coupled with seven amino acids fragment. The resulting nonapeptide may be isolated by precipitation. The material is then subjected to a global side chain deprotection and precipitated with cold MTBE. The resulting peptide is purified in the single pass by reverse phase HPLC and isolated in high yield and purity.

Yet another embodiment of the present invention is to provide a process for the preparation of H-DLeu-Leu-Arg(Pbf)-Pro-NHEt (Fragment-VI) in solution phase by coupling the Fmoc-DLeu-Leu-OH with H-Arg(Pbf)-Pro-NHEt containing coupling reagent at 0-30° C.

In Fmoc solid phase strategy, the tBu group was used to protect the side chains of Ser and Tyr, the trityl group was used to protect the side chains of His and Boc group was used to protect the side chains of Trp amino acid.

### DETAILED DESCRIPTION OF THE INVENTION

The present invention relates to an improved process for large scale production of Leuprolide or its pharmaceutically acceptable salts with increased yield and purity.

For the purpose of clarity and as an aid in the understanding of the invention, as disclosed and claimed herein, the following terms and abbreviations are defined below

The seven amino acids fragment—Pyr-His(Trt)-Trp(Boc)-Ser(tBu)-Tyr(tBu)-DLeu-Leu-OH is represented by Fragment-II

The two amino acids fragment—H-Arg(Pbf)-Pro-NHEt is represented by Fragment-III

The protected Leuprolide—Pyr-His(Trt)-Trp(Boc)-Ser(tBu)-Tyr(tBu)-DLeu-Leu-Arg(Pbf)-Pro-NHEt is represented by Fragment-IV,

The five amino acids fragment—Pyr-His(Trt)-Trp(Boc)-Ser(tBu)-Tyr(tBu)-OH is represented by Fragment-V,

The four amino acids fragment—H-DLeu-Leu-Arg(Pbf)-Pro-NHEt is represented by Fragment-VI.
AcOH acetic acid
tBu tert-butyl
DCC N,N′-dicyclohexyl carbodiimide
DCM dichloromethane
DIC N,N′-diisopropylcarbodiimide
DMF N,N′-Dimethylformamide
DIEA Diisopropylethylamine
2-CTC resin 2-Chlorotrityl chloride resin
DTT Dithiothreitol
Fmoc 9-fluorenylmethoxycarbonyl
6-CI-HOBt 6-Chloro-1-Hydroxybenzotriazole
HOBt N-hydroxybenzotriazole
HBTU O-Benzotriazole-N,N,N′,N′-tetramethyl-uronium-hexafluoro-phosphate
MTBE Methyl tert-butyl ether
Pbf pentmethyldihydrobenzofuransulfonyl
RT room temperature
SPPS solid phase peptide synthesis
Trt trityl
Trp Trptophan
Ser Serine
Tyr Tyrosine
Leu Leucine
His Histidine
Pro Proline
TFA trifluoroacetic acid
EDT ethane dithiol
DDM n-dodecyl 13-D-maltoside
TES Triethylsilane
TIS triisopropylsilane
Pyr Pyrroglutamic acid

Pyr-His-Trp-Ser-Tyr-DLeu-Leu-Arg-Pro-NHEt
1 2 3 4 5 6 7 8 9
Leuprolide (Fragment-I)

One embodiment of the present invention is to provide a process for the preparation of Leuprolide or its pharmaceutically acceptable salts thereof by coupling Pyr-His(Trt)-Trp(Boc)-Ser(tBu)-Tyr(tBu)-DLeu-Leu-OH (Fragment-II) with Fmoc-Arg(Pbf)-Pro-NHEt (Fragment-III) in presence of a coupling reagent to get Pyr-His(Trt)-Trp(Boc)-Ser(tBu)-Tyr(tBu)-DLeu-Leu-Arg(Pbf)-Pro-NHEt (Protected Leuprolide, Fragment-IV), deprotection of Fragment-IV to get Leuprolide, optionally converting into pharmaceutically acceptable salts

According to the present invention, the solvent used to couple the fragments is selected from N,N-dimethylformamide, chloroform, N-methylpyrrolidone, tetrahydrofuran or mixtures thereof.

According to the present invention, the coupling reagent used for the coupling of fragments is selected from N,N′-diisopropylcarbodiimide (DIC)/6-chloro-1-hydroxybenzotriazole(6-CI-HOBO, N,N′-diisopropylcarbodiimide (DIC)/1-Hydroxybenzotriazole(HOBt), 2-(1H-benzotriazol-1-yl)-1,1,3,3-tetramethyl-uronium hexafluorophosphate (HBTU)/HOBt/N,N-diisopropylethylamine(DIEA) or DIC/ethyl 2-cyano-2-(hydroxyimino)acetate (Oxyma).

The coupling of the Fragment-II with Fragment-III is carried out in a range of 0° C. to 30° C.

According to the present invention, the peptide cleavage and global deprotection reagents used in the process of the present invention is a cocktail mixture of acid, scavengers and solvents. Scavengers are selected from EDT, DDM, TIPS, TES, Phenol, thioanisole or mixture thereof.

The reagent for the cleavage of the side chain protecting groups of the Fragment-IV is selected from TFA/EDT/Thioanisole/DCM/TIPS (80%/5%/5%/5%/5%), TFA/EDT/TIS (95%/2.5%/2.5%) or TFA/DTT/Water (95/2.5/2.5).

According to the present invention, after completion of the reaction, the reaction mixture may optionally be filtered and washed with acid or an organic solvent. The crude leuprolide is isolated by combining the reaction mass with an organic solvent, preferably by combining with an ether solvent. The ether solvent is selected from group comprising of diethyl ether, diisopropyl ether, tert-butyl methyl ether, tert-butyl ethyl ether, isopropyl ether or mixture thereof.

Another embodiment of the present invention is to provide a process for the preparation of Leuprolide or its pharmaceutically acceptable salts thereof by Pyr-His(Trt)-Trp(Boc)-Ser(tBu)-Tyr(tBu)-OH (Fragment-V) with H-DLeu-Leu-Arg(Pbf)-Pro-NHEt (Fragment-VI) in presence of a coupling reagent to get Pyr-His(Trt)-Trp(Boc)-Ser(tBu)-Tyr(tBu)-DLeu-Leu-Arg(Pbf)-Pro-NHEt (Protected Leuprolide, Fragment-IV), deprotection of Fragment-IV to get Leuprolide, optionally converting into pharmaceutically acceptable salts.

According to the present invention, the solvent used to couple the fragments is selected from N,N-dimethylformamide, chloroform, N-methylpyrrolidone, tetrahydrofuran or mixtures thereof.

The coupling reagent used for the coupling of fragments is selected from DIC/6-CI-HOBt, DIC/HOBt, HBTU/HOBt/DIEA or DIC/ethyl 2-cyano-2-(hydroxyimino)acetate (Oxyma).

The coupling of the Fragment-V with Fragment-VI is carried out in a range of 0° C. to 30° C.

According to the present invention, the peptide cleavage and global deprotection reagents used in the process of the present invention is a cocktail mixture of acid, scavengers and solvents. Scavengers are selected from EDT, DDM, TIPS, TES, Phenol, thioanisole or mixture thereof.

The reagent for the cleavage of the side chain protecting groups of the Fragment-IV is selected from TFA/EDT/Thioanisole/DCM/TIPS (80%/5%/5%/5%/5%), TFA/EDT/TIS (95%/2.5%/2.5%) or TFA/DTT/Water (95/2.5/2.5).

According to the present invention, after completion of the reaction, the reaction mixture may optionally be filtered and washed with acid or an organic solvent. The crude leuprolide is isolated by combining the reaction mass with an organic solvent, preferably by combining with an ether solvent. The ether solvent is selected from group comprising of diethyl ether, diisopropyl ether, tert-butyl methyl ether, tert-butyl ethyl ether, isopropyl ether or mixture thereof.

According to the present invention, isolation may be carried out by adding an ether solvent to the reaction mass or by adding the reaction mass to the ether solvent selected. Preferably, the reaction mass is added to an ether solvent. More preferably, the reaction mass is added to an ether solvent pre-cooled to a temperature of about −5° C. to 5° C.

Yet another embodiment of the present invention is to provide Pyr-His(Trt)-Trp(Boc)-Ser(tBu)-Tyr(tBu)-DLeu-Leu-OH (Fragment-II).

Yet another embodiment of the present invention is to provide a process for the preparation of Pyr-His(Trt)-Trp(Boc)-Ser(tBu)-Tyr(tBu)-DLeu-Leu-OH (Fragment-II) comprising the steps of:
a) anchoring seventh protected terminal amino acid to a resin,
b) capping the resin obtained in step a),
c) selectively deprotecting the amino group,
d) coupling carboxyl terminus of the next N-protected amino acid to the amine group in presence of a coupling reagent,
e) repeating steps c) and d) to form a peptide sequence,
f) cleaving the peptide from the resin to isolate Fragment-II.

According to the present invention, the resin used for synthesis of peptide undergoes swelling in presence of a solvent selected from dichloromethane, tetrahydrofuran, N,N-dimethylformamide, and N,N-dimethylacetamide, N-methyl-2-pyrrrolidone or mixtures thereof and is then treated with N-terminus protected amino acid and DIEA for a desired period of time for the esterification to the 2-CTC resin.

After anchoring the first protected amino acid to resin, before proceeding to next step, the unreacted linkers on the resin (polymer) are protected (capped) to avoid the undesired peptide chain formation. Then deprotection of the protected amino acid attached to the resin is done selectively in the presence of a nucleophilic base such as 20% piperidine in N,N-dimethylformamide, methylene chloride, tetrahydrofuran or N-methylpyrrolidine and the coupling agents used for the coupling is selected from DIC/6-CI-HOBt, DIC/HOBt, HBTU/HOBt/DIEA or DIC/Oxyma and the solvents used in the coupling reaction is carried out in the presence of solvents selected from group comprising of dichloromethane, tetrahydrofuran, dimethylformamide, N-methylpyrolidone or mixtures thereof. The amount of protected amino acid used in the present invention is selected from 1M to 5 M with respect to resin loading capacity.

According to the present invention, the resin is selected from 2-chlorotrityl chloride resin or ethylamine 2-chlorotrityl resin.

According to the present invention, capping of the functional groups is carried out by using acetic anhydride, pyridine and dichloromethane.

According to the present invention, the resin after the completion of the reaction is optionally washed with solvents such as DMF and DCM to remove residual reagents and byproducts. The process is repeated if desired and before proceeding to next step.

According to the present invention, cleavage of the peptide from the resin carried out by using a 1% TFA in DCM for 5 min.

Yet another embodiment of the present invention is to provide a process for the purification of Pyr-His(Trt)-Trp(Boc)-Ser(tBu)-Tyr(tBu)-DLeu-Leu-OH (Fragment-II) is carried out by dissolving in solvent and adding an anti-solvent to isolated Fragment-II in 80% yield with 83% PHLC purity.

According to the present invention, the solvent is selected from methanol, ethanol, 1-propanol or 2-propanol and the anti-solvent is the selected from chloroform or methylene chloride.

Yet another embodiment of the present invention is to provide H-Arg(Pbf)-Pro-NHEt (Fragment-III).

Yet another embodiment of the present invention is to provide a process for the preparation of Fmoc-Arg(Pbf)-Pro-NHEt (Fragment-III) in solution phase by coupling the Fmoc-Arg(Pbf) with H-Pro-NHEt in presence of a coupling agent at 0-30° C. According to the present invention, the solvent used to couple the amino acids is selected from N,N-dimethylformamide, chloroform, N-methylpyrrolidone, tetrahydrofuran or mixtures thereof.

The coupling reagent used for the coupling of fragments is selected from DIC/6-CI-HOBt, DIC/HOBt, HBTU/HOBt/DIEA or DIC/ethyl 2-cyano-2-(hydroxyimino)acetate (Oxyma).

According to the present invention, the two amino acids fragment (Fragment-III) from the C-terminus of Leuprolide were synthesized using solution phase coupling procedure utilizing Fmoc Arg(Pbf) and Pro-NHEt. The coupling was performed using HBTU/DIEA/6-CI-HOBt.

Yet another embodiment of the present invention is to provide Pyr-His(Trt)-Trp(Boc)-Ser(tBu)-Tyr(tBu)-DLeu-Leu-Arg(Pbf)-Pro-NHEt (Protected Leuprolide) (Fragment-IV).

Yet another embodiment of the present invention is to provide an improved process for the preparation of Pyr-His(Trt)-Trp(Boc)-Ser(tBu)-Tyr(tBu)-DLeu-Leu-Arg(Pbf)-Pro-NHEt (Fragment-IV) either coupling Pyr-His(Trt)-Trp(Boc)-Ser(tBu)-Tyr(tBu)-DLeu-Leu-OH (Fragment-II) with Fmoc-Arg(Pbf)-Pro-NHEt (Fragment-III) in presence of a coupling reagent at 0-30° C. or by coupling Pyr-His(Trt)-Trp(Boc)-Ser(tBu)-Tyr(tBu)-OH (Fragment-V) with H-DLeu-Leu-Arg(Pbf)-Pro-NHEt (Fragment-VI) in presence of a coupling reagent at 0-30° C.

According to the present invention, the solvent used to couple the fragments is selected from N,N-dimethylformamide, chloroform, N-methylpyrrolidone, tetrahydrofuran or mixtures thereof.

The coupling reagent used for the coupling of fragments is selected from DIC/6-CI-HOBt, DIC/HOBt, HBTU/HOBt/DIEA or DIC/ethyl 2-cyano-2-(hydroxyimino)acetate (Oxyma).

Yet another embodiment of the present invention is to provide Pyr-His(Trt)-Trp(Boc)-Ser(tBu)-Tyr(tBu)-OH (Fragment-V).

Yet another embodiment of the present invention is to provide a process for the preparation of Pyr-His(Trt)-Trp(Boc)-Ser(tBu)-Tyr(tBu)-OH (Fragment-V) comprising the steps of:
a) anchoring fifth protected terminal amino acid to a resin,
b) capping the resin obtained in step a),
c) selectively deprotecting of the amino group,
d) coupling carboxyl terminus of the next N-protected amino acid to the amine group in presence of a coupling reagent,
e) repeating steps c) and d) to form a peptide sequence,
f) cleaving the peptide from the resin to isolate Fragment-V.

According to the present invention, the resin used for synthesis of peptide undergoes swelling in presence of a solvent selected from dichloromethane, tetrahydrofuran, N,N-dimethylformamide, and N,N-dimethylacetamide, N-methyl-2-pyrrolidone or mixtures thereof and is then treated with N-terminus protected amino acid and DIEA for a desired period of time for the esterification to the resin.

After anchoring the first protected amino acid to resin, before proceeding to next step, the unreacted linkers on the resin (polymer) are protected (capped) to avoid the undesired peptide chain formation. Then deprotection of the protected amino acid attached to the resin is done selectively in the presence of a nucleophilic base such as 20% piperidine in N,N-dimethylformamide, methylene chloride, tetrahydrofuran or N-methylpyrrolidine and the coupling agents used for the coupling is selected from DIC/6-CI-HOBt, DIC/HOBt, HBTU/HOBt/DIEA or DIC/Oxyma and the solvents used in the coupling reaction is carried out in the presence of solvents selected from group comprising of dichloromethane, tetrahydrofuran, dimethylformamide, N-methylpyrolidone or mixtures thereof. The amount of protected amino acid used in the present invention is selected from 1 M to 5 M with respect to resin loading capacity.

According to the present invention, the resin is selected from 2-chlorotrityl chloride resin or ethylamine 2-chlorotrityl resin.

According to the present invention, capping of the functional groups is carried out by using acetic anhydride, pyridine and dichloromethane.

According to the present invention, the resin after the completion of the reaction is optionally washed with solvents such as DMF and DCM to remove residual reagents and byproducts. The process is repeated if desired and before proceeding to next step. According to the present invention, cleavage of the peptide from the resin carried out by using a 1% TFA in DCM for 5 min.

Yet another embodiment of the present invention is to provide H-DLeu-Leu-Arg(Pbf)-Pro-NHEt (Fragment-VI).

Yet another embodiment of the present invention is to provide a process for the preparation of H-DLeu-Leu-Arg(Pbf)-Pro-NHEt (Fragment-VI) in solution phase by coupling the Fmoc-DLeu-Leu-OH with H-Arg(Pbf)-Pro-NHEt containing coupling reagent at 0-30° C.

According to the present invention, the solvent used to couple the fragments is selected from N,N-dimethylformamide, chloroform, N-methylpyrrolidone, tetrahydrofuran or mixtures thereof.

The coupling reagent used for the coupling of fragments is selected from DIC/6-CI-HOBt, DIC/HOBt, HBTU/HOBt/DIEA or DIC/ethyl 2-cyano-2-(hydroxyimino)acetate (Oxyma).

Yet another embodiment of the present invention is to provide Leuprolide acetate having the purity of more than 98.5% and Pyr-His-Trp-DSer-Tyr-DLeu-Leu-Arg-Pro-NHEt ((4-D-Serine)Leuprolide, Pyr-DHis-Trp-Ser-Tyr-DLeu-Leu-Arg-Pro-NHEt ((2-D-Histidine)Leuprolide), Pyr-His-Trp-Ser-Tyr-Leu-Leu-Arg-Pro-NHEt ((6-L-Leucine)Leuprolide), Pyr-His-Trp-Ser(Ac)-Tyr-DLeu-Leu-Arg-Pro-NHEt ((4-(O-Acetyl-L-Serine))Leuprolide) and Pyr-His-Trp-Ser-Tyr-DLeu-DLeu-Arg-Pro-NHEt ((7-D-leucine)Leuprolide) impurities about 1.50% in total.

Yet another embodiment of the present invention is to provide a process for the purification of protected Leuprolide (Fragment-IV) is carried out by dissolving in solvent and adding an anti-solvent to isolated protected Leuprolide in enriched purity.

According to the present invention, the solvent is selected for the dissolution is selected from ethyl acetate or acetonitrile and anti-solvent is selected from n-hexane, pentane, octane, isopropyl ether or methyl tert-butyl ether.

Yet another embodiment of the present invention is to provide a process for the purification of crude Leuprolide is carried out by dissolving in solvent and adding an anti-solvent to isolated Leuprolide in 85% yield with 80% HPLC purity.

According to the present invention, the solvent is selected from methanol, ethanol, 1-propanol or 2-propanol and the anti-solvent is the selected from n-hexane, pentane, octane, isopropyl ether or methyl tert-butyl ether.

Yet another embodiment of the present invention is to provide a process for the purification of crude Leuprolide by preparative HPLC method in the presence of a buffer and methanol/acetonitrile as eluting agents and isolating pure Leuprolide acetate.

The schematic description of the process for the preparation of Leuprolide is as shown in scheme-I:

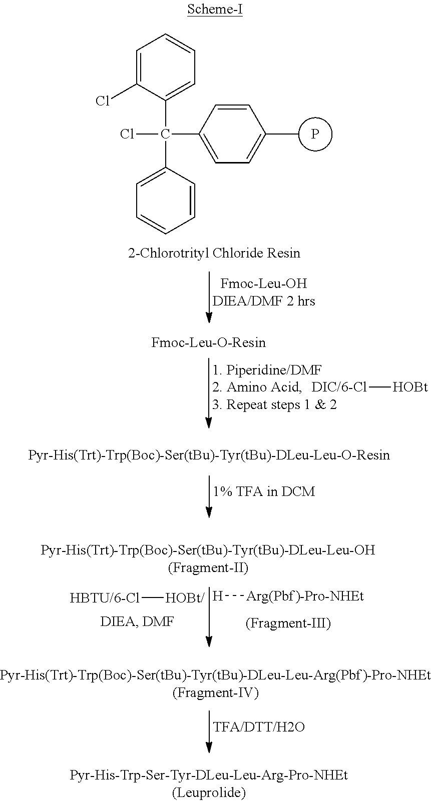

The schematic description of the process for the preparation of Fragment-III is as shown in scheme-II:

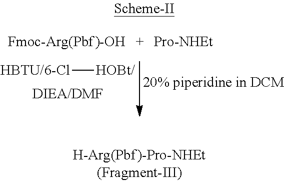

The schematic description of the process for the preparation of Leuprolide is as shown in scheme-III.

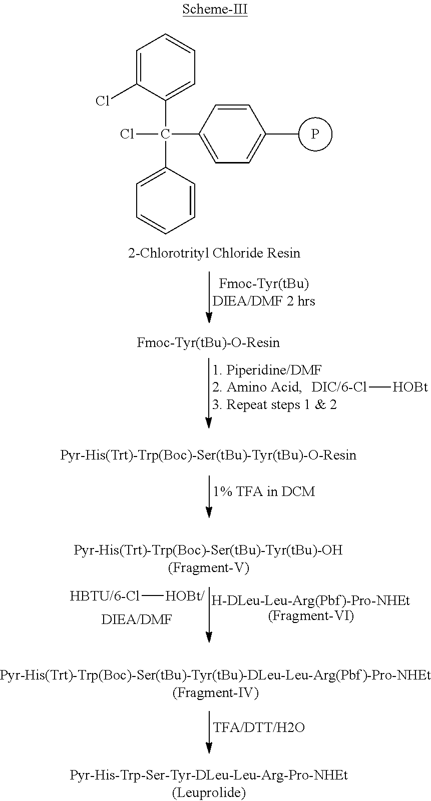

The schematic description of the process for the preparation of Fragment-IV is as shown in scheme-IV

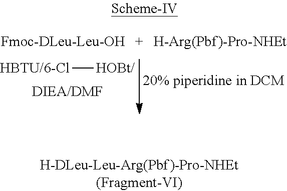

The invention is illustrated with the following examples, which are provided by way of illustration only and should not be construed to limit the scope of the invention.

### Example-1

### Solid phase synthesis of Pyr-His(Trt)-Trp(Boc)-Ser(tBu)-Tyr(tBu)-DLeu-Leu-OH using 2-CTC resin (Fragment-II)

Synthesis of the peptide was carried out by a regular stepwise Fmoc SPPS procedure starting from 2-chlorotrityl chloride resin (10 g, loading 0.7 mmol/g) on CS Bio fully automated solid phase peptide synthesizer. The resin was swelled in dichloromethane (50 ml) for 2 hrs followed by DMF (50 ml) for 2 hrs. The attachment of first amino acid Fmoc-Leu (6.8 g, 19.3 mmol) and diisopropylethylamine (DIEA, 14 mL, 78.6 mmol) were added. The mixture was stirred at room temperature for 2 hrs, filtered and washed with DMF and DCM. Subsequently, the mixture was stirred with a solution of 5% DIEA and 10% methanol in DCM for 30 min. The resin was washed with DMF and DCM and dried in vacuum to yield the loaded resin. The loading was determined using Beer-Lambert law and found to be 0.5 mmol/g. After resin loading and prior to first deprotection, the resin is allowed to swell in DMF for 1 hr. The Fmoc group was deprotected with 20% piperidine in DMF and subsequent amino acid couplings were done using DIC/6-CI-HOBt as coupling agent. After every coupling and deprotection through washings were done prior to proceed for next amino acid. All the couplings and deprotections were monitored using ninhydrin test. The cleaving the peptide from the resin is carried with 1% TFA in DCM.

### Example-2

### Solution phase synthesis of H-Arg(Pbf)-Pro-NHEt (Fragment-III)

To a stirred solution of Fmoc-Arg(Pbf) (1.0 eq) and 6-CI-HOBt (1.2 eq) in DMF, was added DIEA (3.0 eq) and cooled to 10° C. HBTU (1.0 eq) was added to the reaction mixture, stirred for 15 min. H-Pro-NHEt (1.0 eq) (The HCl salt was neutralized with DIEA prior to the addition) was added and stirred for over night at room temperature. The reaction mixture was cooled and then water was added, stirred for about 30 min. The precipitated solid was filtered, washed with water and dried under vacuum. The material was washed with IPE and dried. Then Fmoc group is deprotected with 20% DCM/piperidine for 30 min and precipitated with IPE and further carried for next coupling.

### Example-3

### Solution phase synthesis of Pyr-His(Trt)-Trp(Boc)-Ser(tBu)-Tyr(tBu)-DLeu-Leu-Arg(Pbf)-Pro-NHEt (Protected Leuprolide) (Fragment-IV) by 7+2 fragment condensation method

To a stirred solution of Fragment-II (1.0 eq) and 6-CI-HOBt (1.2 eq) in DMF, was added DI EA (3.0 eq) and cool to 10° C. HBTU (1.0 eq) was added to the reaction mixture, stirred for 15 min at 10° C., then H-Arg(Pbf)-Pro-NHEt (Fragment-III, 1.0 eq) was added and stirred for over night at room temperature. The reaction mixture was cooled and then water was added, stir for 30 min. The pecipitated solid was filtered, washed with water and dried under vacuum. The material was washed with IPE and dried.

### Example-4

### Solid Phase Synthesis of Pyr-His(Trt)-Trp(Boc)-Ser(tBu)-Tyr(tBu)-OH using 2-CTC Resin (Fragment-V)

Synthesis of the peptide was carried out by a regular stepwise Fmoc SPPS procedure starting from 2-chlorotrityl chloride resin (10 g, loading 0.7 mmol/g) on CS Bio fully automated solid phase peptide synthesizer. The resin was swelled in dichloromethane (50 ml) for 2 hours followed by DMF (50 ml) for 2 hrs. The attachment of first amino acid Fmoc-Tyr(tBu) (2.0 eq) and diisopropylethylamine (DIEA, 14 mL, 78.6 mmol) were added. The mixture was stirred at room temperature for about 2 hrs, filtered and washed with DMF and DCM. Subsequently, the mixture was stirred with a solution of 5% DIEA and 10% methanol in DCM for 30 min. The resin was washed with DMF and DCM and dried in vacuum to yield the loaded resin. The loading was determined using Beer-Lambert law and found to be 0.5 mmol/g. After resin loading and prior to first deprotection, the resin is allowed to swell in DMF for about 1 hr. The Fmoc group was deprotected with 20% piperidine in DMF and subsequent amino acid couplings were done using DIC/6-CI-HOBt as coupling agent. After every coupling and deprotection through washings were done prior to proceed for next amino acid. All the couplings and deprotections were monitored using ninhydrin test. The cleaving the peptide from the resin is carried with 1% TFA in DCM.

### Example-5

### Solution phase synthesis of Fmoc-DLeu-Leu-OH

Fmoc-DLeu (1 eq) was dissolved in THF (10 ml) was added DCC (1 eq) and HOSu (1.2 eq). The mixture was stirred at room temperature for 5 hrs. The DCU was filtered and added H-Leu-OH (1.5 eq) dissolved in 10% NaHCO3 (10 ml) and stirred overnight at RT. The THF was evaporated and extracted the aqueous layer with ethyl acetate. It was evaporated and precipitated with hexane gives white crystalline solid.

### Example-6

### Solution phase synthesis of H-DLeu-Leu-Arg(Pbf)-Pro-NHEt (Fragment-VI)

To a stirred solution of Fmoc-DLeu-Leu-OH (1.0 eq) and 6-CI-HOBt (1.2 eq) in DMF, was added DIEA (3.0 eq) and cool to 10° C. HBTU (1.0 eq) was added to the reaction mixture, stirred for 15 min at 10° C., then H-Arg(Pbf)-Pro-NHEt (1.0 eq) was added and stirred for over night at room temperature. The reaction mixture was cooled and then water was added, stirred for 30 min. The precipitated solid was filtered, washed with water and dried under vacuum. The material was washed with IPE and dried.

### Example-7

### Solution phase synthesis of Pyr-His(Trt)-Trp(Boc)-Ser(tBu)-Tyr(tBu)-DLeu-Leu-Arg(Pbf)-Pro-NHEt (Protected Leuprolide) (Fragment-IV) by 5+4 fragment condensation method

To a stirred solution of Pyr-His (Trt)-Trp(Boc)-Ser(tBu)-Tyr(tBu)-OH (Fragment-V, 1.0 eq) and 6-CI-HOBt (1.2 eq) in DMF, was added DIEA (3.0 eq) and cool to 10° C. HBTU (1.0 eq) was added to the reaction mixture, stirred for 15 min at 10° C., then H-DLeu-Leu-Arg(Pbf)-Pro-NHEt (Fragment-VI, 1.0 eq) was added and stirred for over night at room temperature. The reaction mixture was cooled and then water was added, stir for 30 min. The precipitated solid was filtered, washed with water and dried under vacuum. The material was washed with IPE and dried.

### Example-8

### Preparation of crude Leuprolide (Global side chain deprotection of Fragment-IV)

Deprotection of the side chain functional groups of leuprolide are cleaved using Cocktail consisting of TFA/EDT/Thioanisole/DCM/TIPS in the preferred volumes of 80%/5%/5%/5%/5% the cocktail was cooled and a solution of protected peptide in DCM was added at 10° C. The resultant reaction mass was stirred for 2 h at room temperature, cooled, the chilled MTBE was added and stirred for 30 min. The solid was filtered and washed with Ethyl acetate and MTBE.

### Example-9

### Purification of Leuprolide

The crude Leuprolide was loaded on to preparative C18 column (50×250 mm, 100A°). The peptide was purified using aqueous acetic acid (0.05%) and 5% methanol in acetonitrile using gradient method. The pure fraction containing the Leuprolide was pooled. The methanol was evaporated and the aqueous layer was lyophilized to give the Leuprolide as white solid. The purified Leuprolide was analyzed by RP-HPLC and mass determined by mass spectrometer.

1-22. (canceled)

23. A process for the preparation of Leuprolide or a pharmaceutically acceptable salt thereof comprising the steps of:
coupling Pyr-His(Trt)-Trp(Boc)-Ser(tBu)-Tyr(tBu)-DLeu-Leu-OH (Fragment-II) with Fmoc-Arg(Pbf)-Pro-NHEt (Fragment-III) in the presence of a coupling reagent to get Pyr-His(Trt)-Trp(Boc)-Ser(tBu)-Tyr(tBu)-DLeu-Leu-Arg(Pbf)-Pro-NHEt (Protected Leuprolide, Fragment-IV);
deprotecting said Fragment-IV to get Leuprolide; and
optionally converting said Leuprolide into a pharmaceutically acceptable salt.

24. A process for the preparation of Leuprolide or a pharmaceutically acceptable salt thereof comprising the steps of:
coupling Pyr-His(Trt)-Trp(Boc)-Ser(tBu)-Tyr(tBu)-OH (Fragment-V) with H-DLeu-Leu-Arg(Pbf)-Pro-NHEt (Fragment-VI) in the presence of a coupling reagent to get Pyr-His(Trt)-Trp(Boc)-Ser(tBu)-Tyr(tBu)-DLeu-Leu-Arg(Pbf)-Pro-NHEt (Protected Leuprolide, Fragment-IV);
deprotecting said Fragment-IV to get Leuprolide; and
optionally converting said Leuprolide into a pharmaceutically acceptable salt.

25. The process according to claim 23 or 24, wherein said deprotection is carried out by using a reagent selected from the group consisting of TFA/EDT/Thioanisole/DCM/TIPS (80%/5%/5%/5%/5%), TFA/EDT/TIS (95%/2.5%/2.5%), and TFA/DTT/Water (95%/2.5%/2.5%).

26. The process according to claim 23, wherein said Pyr-His(Trt)-Trp(Boc)-Ser(tBu)-Tyr(tBu)-DLeu-Leu-OH (Fragment-II) is prepared by a process comprising the steps of:
a) anchoring a seventh protected terminal amino acid to a resin;
b) capping the resin obtained in step a);
c) selectively deprotecting an amino group of the amino acid;
d) coupling a carboxyl terminus of a next N-protected amino acid to the amine group in the presence of a coupling reagent;
e) repeating steps c) and d) to form a peptide sequence; and
f) cleaving the peptide sequence from the resin to isolate Fragment-II.

27. The process according to claim 24, wherein said Pyr-His(Trt)-Trp(Boc)-Ser(tBu)-Tyr(tBu)-OH (Fragment-V) is prepared by a process comprising the steps of:
a) anchoring a fifth protected terminal amino acid to a resin;
b) capping the resin obtained in step a);
c) selectively deprotecting an amino group of the amino acid;
d) coupling the carboxyl terminus of a next N-protected amino acid to the amine group in the presence of a coupling reagent;
e) repeating steps c) and d) to form a peptide sequence; and
f) cleaving the peptide from the resin to isolate Fragment-V.

28. The process according to claim 26 or 27, wherein said resin is selected from the group consisting of 2-chlorotrityl chloride resin and ethylamine 2-chlorotrityl resin.

29. The process according to claim 26 or 27, wherein said cleavage of the peptide from the resin is carried out with 1% trifluoroacetic acid (TFA) in dichloromethane (DCM).

30. The process according to claim 23, wherein said Fmoc-Arg(Pbf)-Pro-NHEt (Fragment-III) is prepared by coupling Fmoc-Arg(Pbf) with H-Pro-NHEt in the presence of a coupling agent.

31. The process according to claim 24, wherein said H-DLeu-Leu-Arg(Pbf)-Pro-NHEt (Fragment-VI) is prepared by coupling Fmoc-DLeu-Leu-OH with H-Arg(Pbf)-Pro-NHEt in the presence of a coupling reagent.

32. The process according to claim 23, 24, 26, 27, 30 or 31, wherein said coupling reagent is selected from the group consisting of
N,N′-diisopropylcarbodiimide (DIC)/6-chloro-1-hydroxybenzotriazole (6-CI-HOBt),
N,N′-diisopropylcarbodiimide (DIC)/1-hydroxybenzotriazole (HOBt),
2-(1H-benzotriazol-1-yl)-1,1,3,3-tetramethyluronium hexafluorophosphate (HBTU)/1-hydroxybenzotriazole (HOBt)/N,N-diisopropylethylamine (DIEA), and
N,N′-diisopropylcarbodiimide (DIC)/ethyl 2-cyano-2-(hydroxyimino)acetate (Oxyma).

33. Leuprolide acetate having a purity of more than 98.5%.

34. A process for the purification of raw Pyr-His(Trt)-Trp(Boc)-Ser(tBu)-Tyr(tBu)-DLeu-Leu-OH (Fragment-II), raw protected Leuprolide (Fragment-IV), or raw crude Leuprolide, comprising the steps of dissolving the raw compound in a solvent and adding an anti-solvent.

35. The process according to claim 34 for the purification of raw Pyr-His(Trt)-Trp(Boc)-Ser(tBu)-Tyr(tBu)-DLeu-Leu-OH (Fragment-II) or raw crude Leuprolide, wherein the solvent is selected from the group consisting of methanol, ethanol, 1-propanol, and 2-propanol.

36. The process according to claim 34 for the purification of raw Pyr-His(Trt)-Trp(Boc)-Ser(tBu)-Tyr(tBu)-DLeu-Leu-OH (Fragment-II), wherein the anti-solvent is the selected from the group consisting of chloroform and methylene chloride.

37. The process according to claim 34 for the purification of raw protected Leuprolide (Fragment-IV), wherein said solvent is selected from the group consisting of ethyl acetate and acetonitrile.

38. The process according to claim 34 for the purification of raw protected Leuprolide (Fragment-IV) or raw crude Leuprolide, wherein said anti-solvent is selected from the group consisting of n-hexane, pentane, octane, isopropyl ether, and methyl tert-butyl ether.
